(19) United States
(12) Patent Application Publication
(10) Pub. No.: US 2025/0177678 A1
(43) Pub. Date: Jun. 5, 2025

(54) VENTILATORY SUPPORT DEVICES FOR USERS WITH RESPIRATORY DISTRESS

(71) Applicants: Neil David Wainwright, Toronto (CA); Erik Kolbjorn Lloyd, Dundas (CA); University Health Network, Toronto (CA)

(72) Inventors: Neil David Wainwright, Toronto (CA); Erik Kolbjorn Lloyd, Dundas (CA); Shafique Keshavjee, Toronto (CA); Marcelo Cypel, Toronto (CA); Raymond Paul Joseph Janisse, Toronto (CA)

(21) Appl. No.: 18/845,259

(22) PCT Filed: Mar. 9, 2023
(86) PCT No.: PCT/CA2023/050307

§ 371 Date: Sep. 9, 2024

Related U.S. Application Data
(60) Provisional application No. 63/318,270, filed on Mar. 9, 2022.

(51) Int. Cl. A61M 16/12; A61M 16/10; A61M 16/20

(52) U.S. Cl. CPC A61M 16/12; A61M 16/1005; A61M 16/208; A61M 2202/0275

(57) ABSTRACT

A device for delivering at least one gas to a user is described herein. The device includes a body defining: a chamber to receive the gas, an inlet leading into the chamber and an outlet for the user to draw the gas from the chamber. The device also includes a demand valve assembly configured to supply the gas to the chamber. The demand valve assembly includes an intake block, at least one demand valve coupled to the intake block and an actuator positioned within the chamber and coupled to the demand valve. The actuator is configured to move the demand valve into an open position to supply gas to the chamber. The device also includes a diaphragm assembly. The diaphragm assembly includes a diaphragm membrane abutting the actuator and movable downwardly relative to the body when the user draws the gas from the chamber to supply gas to the chamber.

### CROSS-REFERENCE

The present application claims the benefit of U.S. Provisional Patent Application No. 63/318,270 entitled “Ventilatory Support Devices for Users with Respiratory Distress” filed Mar. 9, 2022, the entire contents of which are hereby incorporated by reference herein.

### FIELD

This disclosure relates generally to ventilatory support devices, and more specifically to ventilatory support devices for delivering gases to users with respiratory distress.

### BACKGROUND

Nitric oxide (NO) is an important signaling molecule between cells and is involved in a wide range of processes. Previous research has shown that NO has a direct positive effect on the body's antiviral and immunological defense properties critical to combating viruses. High doses of NO (for example, greater than about 160 ppm) exerts antimicrobial activity against several bacteria, protozoa and some viruses.

Specifically, inhaled NO at high doses can inhibit in vitro replication of the SARS-COV virus by two different mechanisms. First, inhaled NO causes reduced interactions between S proteins and ACE2 receptors. Second, NO inhibits the production of viral RNA due to the nitrosylation of viral proteins.

Based on the genetic similarities between SARS-COV and SARS-COV-2, similar effects of NO on SARS-COV-2 can be hypothesized. Evidently, there is a strong rationale to use inhaled NO for therapeutic use.

Furthermore, there are additional ways in which NO affects the pathogenesis of viral infections. First, NO, when administered in high doses by inhalation, suppresses different stages of viral reproduction due to the nitrosylation of viral proteins. This inhibits virus spread and enables pathogen clearance by the immune system. NO is known to have an antiviral effect on DNA and RNA-based viruses, including some types of coronaviruses.

Second, NO acts as an anti-inflammatory immune response modulator. Depending on the type of virus, Th1 and Th2 cells protect the body from viral pathogens. Overproduction of NO suppresses Th1 immune responses, leading to a Th2-biased immunoregulatory balance.

Unfortunately, there are known risks of administering NO to patients. For example, it is also known that high doses of inhaled NO may contribute to oxidative stress, which can damage tissues if administration is not tightly controlled. Another important consideration is preventing high levels of NO₂ (nitric dioxide) when administering NO and oxygen (O₂) together.

Currently, there are no suitable non-invasive ventilation techniques that enable the dual delivery of therapeutic gases, such as but not limited to NO and O₂, together continuously from high pressure sources to patients with respiratory distress.

One example of patients in need of such a device are patients suffering from respiratory distress caused by COVID-19. Current ventilator approaches are highly invasive and involve intubating COVID-19 patients who require oxygen support. With ventilator demand already high, supply will continue to be limited and rationed. Additionally, an easy-to-use ventilatory support device that could deliver a therapeutic agent with the augmented oxygen could serve as a life-saving intervention.

Therefore, there is a need for new ventilatory support devices, and more specifically to ventilatory support devices for delivering gases to patients with respiratory distress.

### SUMMARY

In accordance with a broad aspect, a device for delivering at least one gas to a user is described herein. The device includes a body defining: a chamber to receive the gas from a source; an inlet leading into the chamber; and an outlet for the user to draw the gas from the chamber. The device also includes a demand valve assembly configured to supply the gas to the chamber via the inlet. The demand valve assembly includes an intake block secured to the body; at least one demand valve coupled to the intake block and configured to control the supply of gas to the chamber; and an actuator positioned within the chamber and coupled to the demand valve, the actuator being configured to move the demand valve into an open position to supply gas to the chamber. The device also includes a diaphragm assembly coupled to the body, the diaphragm assembly including a diaphragm membrane abutting the actuator and movable downwardly relative to the body when the user draws the gas from the chamber to move the demand valve into the open position to supply gas to the chamber.

In at least one embodiment, the demand valve assembly includes a first demand valve coupled to a first gas source to supply a first gas to the chamber and a second demand valve coupled to a second gas source to supply a second gas to the chamber, each demand valve being coupled to the actuator.

In at least one embodiment, the actuator is configured to simultaneously move each of the demand valves to their respective open positions to supply each of the gases to the chamber.

In at least one embodiment, the actuator is coupled to a stem of each of the demand valves.

In at least one embodiment, each of the demand valves includes a biasing member that biases the demand valves to a closed position.

In at least one embodiment, the diaphragm membrane includes a rigid portion configured to abut the actuator.

In at least one embodiment, the rigid portion is positioned in a center of the diaphragm membrane.

In at least one embodiment, the actuator abuts the rigid portion of the diaphragm membrane.

In at least one embodiment, the diaphragm membrane is made of a polymeric material configured to provide the demand valve with a cracking pressure suitable to inhalation capabilities of a user with respiratory distress.

In at least one embodiment, the diaphragm membrane is made of a polymeric material configured to provide for the demand valve to have a cracking pressure in a range of about −1 cm H₂O to about −3 cm H₂O.

In at least one embodiment, the device further includes an input assembly configured to receive two or more gases from respective sources, the input assembly being coupled to the demand valve assembly for providing the gases to the chamber.

In at least one embodiment, the input assembly comprises a preliminary chamber configured to receive two or more gases from respective sources and provide for the two or more gases to mix with each other prior to being provided to the chamber.

In at least one embodiment, the input assembly comprises two one-way valves, each one-way valve for receiving one of the two or more gases and inhibiting the two or more gases from exiting the preliminary chamber.

These and other features and advantages of the present application will become apparent from the following detailed description taken together with the accompanying drawings. It should be understood, however, that the detailed description and the specific examples, while indicating preferred embodiments of the application, are given by way of illustration only, since various changes and modifications within the spirit and scope of the application will become apparent to those skilled in the art from this detailed description.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

FIG. 1 is a perspective view of a ventilatory support device, according to at least one embodiment described herein.

FIG. 2A is a side view showing internal structures of the ventilatory support device of FIG. 1 when the actuator is in the closed position.

FIG. 2B is a side view showing internal structures of the ventilatory support device of FIG. 1 when the actuator is in the open position.

FIG. 3 is an exploded view of the ventilatory support device of FIG. 1.

FIG. 4A is a front perspective view of an intake block of the device of FIG. 1.

FIG. 4B is rear perspective view of the intake block of the device of FIG. 1.

FIG. 4C is a cross-sectional view of the intake block of the device of FIG. 1.

FIG. 5 is a perspective view of another ventilatory support device, according to at least one embodiment described herein.

FIG. 6 is a side view showing internal structures of the ventilatory support device of FIG. 5.

FIG. 7A is a top-down view of the ventilatory support device of FIG. 5.

FIG. 7B is a front view of the ventilatory support device of FIG. 5.

FIG. 8 is an exploded view of the ventilatory support device of FIG. 5.

Further aspects and features of the example embodiments described herein will appear from the following description taken together with the accompanying drawings.

### DESCRIPTION OF EXAMPLE EMBODIMENTS

Various apparatuses, methods and compositions are described below to provide an example of at least one embodiment of the claimed subject matter. No embodiment described below limits any claimed subject matter and any claimed subject matter may cover apparatuses and methods that differ from those described below. The claimed subject matter are not limited to apparatuses, methods and compositions having all of the features of any one apparatus, method or composition described below or to features common to multiple or all of the apparatuses, methods or compositions described below. It is possible that an apparatus, method or composition described below is not an embodiment of any claimed subject matter. Any subject matter that is disclosed in an apparatus, method or composition described herein that is not claimed in this document may be the subject matter of another protective instrument, for example, a continuing patent application, and the applicant(s), inventor(s) and/or owner(s) do not intend to abandon, disclaim, or dedicate to the public any such invention by its disclosure in this document.

Furthermore, it will be appreciated that for simplicity and clarity of illustration, where considered appropriate, reference numerals may be repeated among the figures to indicate corresponding or analogous elements. In addition, numerous specific details are set forth in order to provide a thorough understanding of the example embodiments described herein. However, it will be understood by those of ordinary skill in the art that the example embodiments described herein may be practiced without these specific details. In other instances, well-known methods, procedures, and components have not been described in detail so as not to obscure the example embodiments described herein. Also, the description is not to be considered as limiting the scope of the example embodiments described herein.

It should be noted that terms of degree such as “substantially”, “about” and “approximately” as used herein mean a reasonable amount of deviation of the modified term such that the end result is not significantly changed. These terms of degree should be construed as including a deviation of the modified term, such as 1%, 2%, 5%, or 10%, for example, if this deviation does not negate the meaning of the term it modifies.

Furthermore, the recitation of any numerical ranges by endpoints herein includes all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about” which means a variation up to a certain amount of the number to which reference is being made, such as 1%, 2%, 5%, or 10%, for example, if the end result is not significantly changed.

It should also be noted that, as used herein, the wording “and/or” is intended to represent an inclusive—or. That is, “X and/or Y” is intended to mean X, Y or X and Y, for example. As a further example, “X, Y, and/or Z” is intended to mean X or Y or Z or any combination thereof. Also, the expression of A, B and C means various combinations including A; B; C; A and B; A and C; B and C; or A, B and C.

The following description is not intended to limit or define any claimed or as yet unclaimed subject matter. Subject matter that may be claimed may reside in any combination or sub-combination of the elements or process steps disclosed in any part of this document including its claims and figures. Accordingly, it will be appreciated by a person skilled in the art that an apparatus, system or method disclosed in accordance with the teachings herein may embody any one or more of the features contained herein and that the features may be used in any particular combination or sub-combination that is physically feasible and realizable for its intended purpose.

Recently, there has been a growing interest in developing new ventilatory support devices, particularly for users with respiratory distress.

In at least one embodiment described herein, a patient-centered, non-invasive ventilatory support device is described.

In at least one embodiment described herein, a ventilatory support device is described that may, for example, be used to assist in providing oxygen treatment to users in respiratory distress, such as but not limited to users suffering from respiratory distress caused by COVID-19.

In at least one embodiment described herein, a ventilatory support device is described that provides for oxygen (O₂) and at least one additional gaseous product (e.g., nitric oxide (NO) to be provided to a user, such as but not limited to a user with severe oxygenation needs, in a controlled hospital setting.

In at least one embodiment described herein, a ventilatory support device is described herein that provides an easy-to-apply, non-invasive oxygen treatment option to patients, or users.

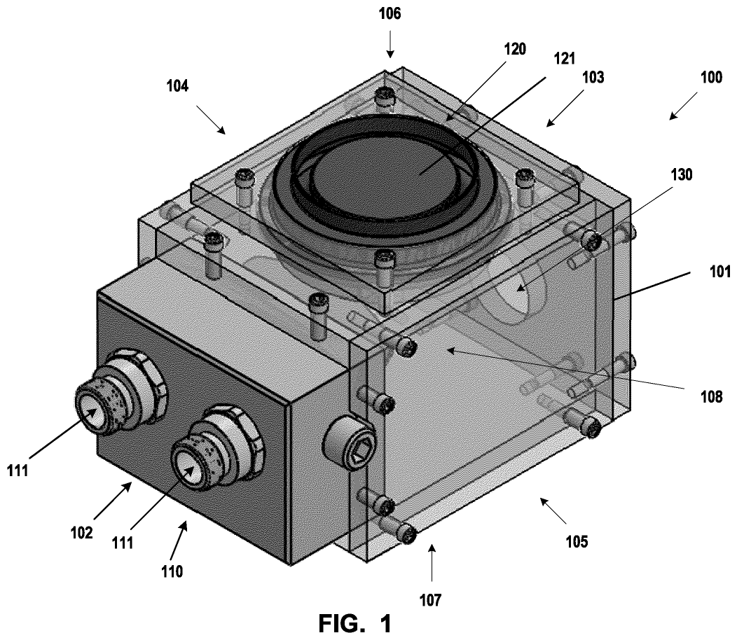

Referring now to FIG. 1, shown therein is a perspective view of a respiratory support device 100 according to at least one embodiment described herein. As noted above, device 100 may be used as a ventilatory support device for a user in respiratory distress.

Body 101 generally has a front 102, a back 103, two opposed sides 104, 105, a top 106 and a bottom 107 to co-operate to form a chamber 108 therein. In the embodiments shown in the drawings, body 101 generally has a cuboid shape, however, body 101 may have any other three-dimensional shape that provides a cavity or chamber therein for the exchange of air from a source and/or environment. In at least one embodiment, body 101 co-operates with intake block 112 and/or diaphragm assembly 120 to define chamber 108.

Device 100 includes a demand valve assembly 110 coupled to the body 101 and configured to provide for the gas(es) to enter the chamber, a diaphragm assembly 120 coupled to the body 101 and configured to control opening and closing of valve(s) of a demand valve assembly 110, and an outlet 130 configured for a patient (or a user) to draw the gas(es) from the chamber 108 when breathing. The demand valve assembly 110 is supplied from a relatively high-pressure source such as a compressed air tank (not shown).

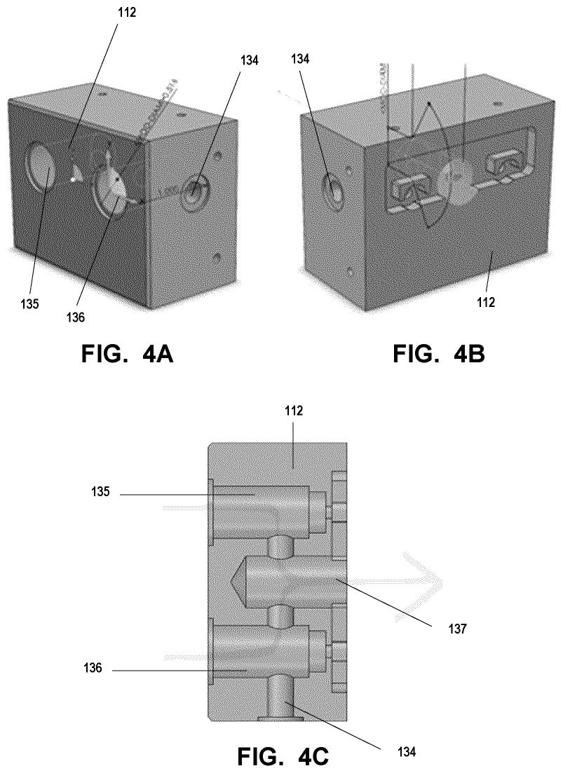

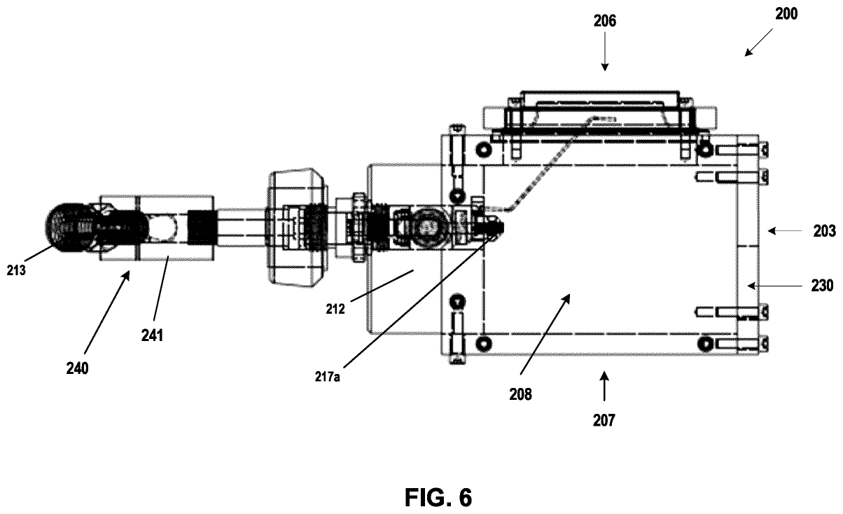

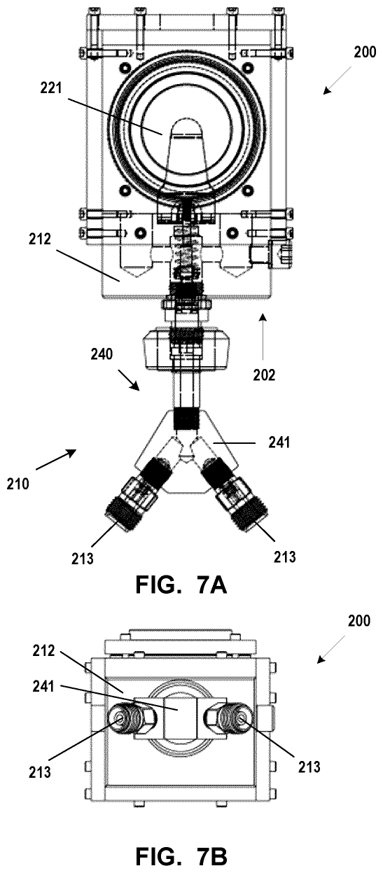

In the embodiment shown in FIGS. 1-3, device 100 can be referred to as dual valve device because demand valve assembly 110 therein includes two demand valves 111 extending outwardly from an intake block 112 that is coupled to body 101. In the embodiment shown in FIGS. 4-7, described in greater detail below, device 200 can be referred to as single valve device because demand valve assembly 210 therein includes a single demand valve 211 extending outwardly from an intake block 212 on front 202 of body 201. For clarity, like components of the two devices shown in the figures will be referred to using same reference numerals, where the reference numbers used in reference to the embodiment shown in FIGS. 4-7 will be increased by 100 relative to the reference numerals used in reference to the embodiment shown in FIGS. 1-3.

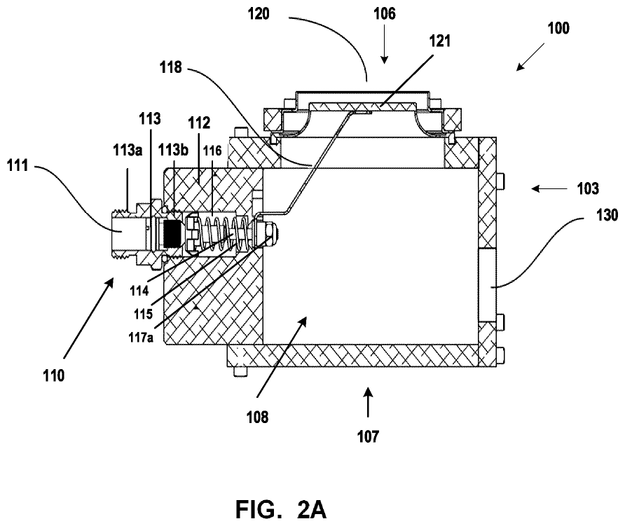

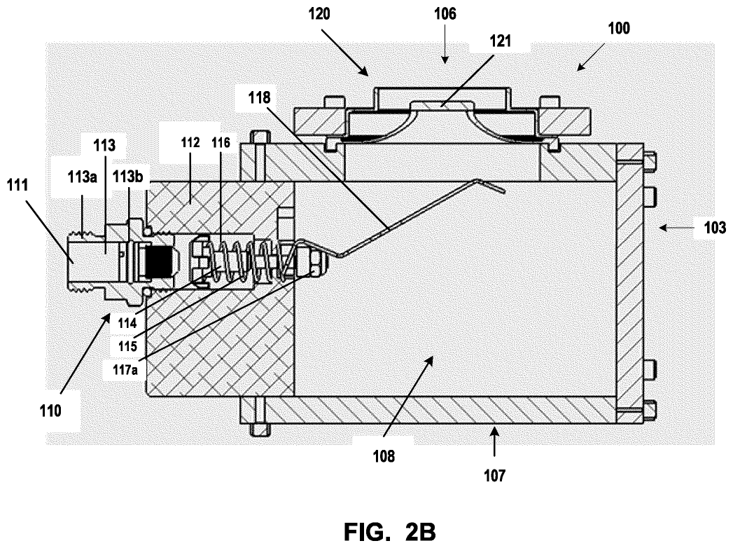

Turning to FIG. 2, shown there is a side view of the device 100 showing the demand valve assembly 110 when each of the demand valves 111 is in a closed position. Demand valves 111 are used as inputs for providing a gas, such as but not limited to oxygen (O₂) and/or nitric oxide (NO), to chamber 108. It should be understood that other gases, including but not limited to therapeutic gases other than O₂ and NO, may also be provided to chamber 108 by valves 110.

Demand valves 111 each have a first member 113 having a first end 113a configured to connect to a source of gas (not shown) and a second member 113b configured to secure the first member 113 to the intake block 112. For example, as shown in FIG. 2, an outer surface of the first end 113a may be threaded to receive a hose, for example, to connect a source of gas to the device 100. For example, as shown in FIG. 2, an outer surface of the second end 113b may also be threaded to be coupled to corresponding threads of intake block 112. Demand vales 111 also include a stem 114 and a biasing element (e.g., a spring) 115. Biasing element 115 biases the stem 114 towards the first member 113 to maintain the demand valve 111 in a closed position. Stem 114 is positioned within a slot 116 or aperture of intake block 112 and configured to slide longitudinally with respect to a longitudinal axis of the device to move between the open and closed positions. In order to open the valve 111, biasing element 115 must be overcome. In the open position, gas(es) may enter the chamber 108 through the intake block 112. Demand valve 111 also includes an adjustable fastener, such as nut 117, that is threadingly received on stem 114 and provides for manually adjusting the biasing force of biasing element 115.

Demand valve assembly 110 also includes an actuator 118 coupled to at least one demand valve 111 (e.g., to stem 114 via nuts 117a and washers 117b). For example, actuator 118 may be in the form of a lever. In the embodiment shown in FIGS. 1-3, actuator 118 is configured to couple to each of the demand valves 111 of demand valve assembly 110 and simultaneously move each of the demand valves 111 towards their open position during actuation.

In at least one embodiment, each of the demand valves 111 may be coupled to a different type of gas to provide for mixing of gases in the chamber 108 prior to inhalation by the user.

Body 101 also includes a diaphragm assembly 120. In the embodiments shown in the figures, diaphragm assembly 120 is positioned on top 108 of body 101. Generally, diaphragm assembly 120 includes a thin elastomeric diaphragm membrane 121 and is used to control the movement of demand valves 111 and, therefore, the supply of gas into chamber 108. As the user withdraws air from the chamber 108, the air pressure in the chamber 108 reduces, causing inward movement of the diaphragm membrane 121 of diaphragm assembly 120. Actuator 118 is thereby caused to move downwards and outwards towards the first member to thereby open the at least one demand valve 111 coupled thereto.

Under equilibrium conditions, the pressure in the chamber 108 equals the pressure of the environment external to the device 100. By substantially equalizing the pressure of supplied air to that of the environment, the user's breathing effort is minimized.

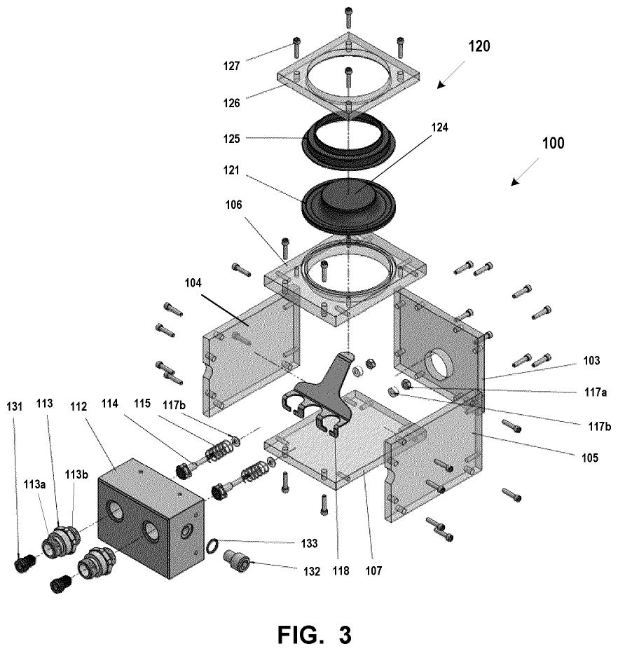

In at least one embodiment, the diaphragm membrane 121 abuts actuator 118 and presses against the actuator 118 to control the opening of demand valve 111. In at least one embodiment, diaphragm membrane 121 of the diaphragm assembly 120 includes a rigid portion 124 (see FIG. 3). In at least one embodiment, the rigid portion 124 is positioned at a center of the diaphragm membrane 121 and abuts actuator 118 and presses against the actuator 118 to control the opening of demand valve 111.

Diaphragm membrane 121 is generally made of a polymeric or other suitable elastomeric material. In at least one embodiment, diaphragm membrane 121 is configured to provide demand valve 111 with a cracking pressure suitable to inhalation capabilities of patients or users with respiratory distress. Herein, the term “cracking pressure” is used to describe the minimum upstream pressure required to open demand valve 111 enough to provide for the gas to enter chamber 108. For example, diaphragm membrane 121 provides for demand valve 111 to have a cracking pressure close to neutral pressure across the membrane. In at least one embodiment, diaphragm membrane 121 is configured to provide for demand valve 111 to have a cracking pressure in a range of about 1 cm H₂O to about 3 cm H₂O. It should be understood that this cracking pressure could also be understood as a negative value as a negative pressure (i.e. −1 cm H₂O to about −3 cm H₂O) is created in the circuit to open the valve.

Referring to FIG. 3, diaphragm assembly 120 may include a peripheral rim 125 configured to fit within an opening of body 101 and sealingly retain diaphragm membrane 121 in body 101. Diaphragm 120 may be clamped in place by the aid of a cap or cover 126 and/or held in place by fasteners 127.

It should be noted that in FIG. 3, back 103, two opposed sides 104, 105, top 106 and bottom 107 are formed by a back plate 103, two opposed side plates 104, 105, a top plate 106 and a bottom plate 107, respectively, each of which is coupled to form body 101 by a plurality of fasteners. Each of the plates, or walls, of body 101 are tightly pressed against each other to form a single unit. Generally, gaskets are not needed to form body 101, but all openings into chamber 108 are sealed by, for example, an o-ring (not shown) at, for example, second end 113b of first member 113. Also, the valve end of stem 114 may have a soft surface that presses against demand valve 111. Intake block 111 is coupled to each of the opposed side plates 104, 105 as well as top plate 106 by a plurality of fasteners.

It should be understood that demand valve 111 is biased to its closed position, so when a user of the device stops breathing, or exhales, neutral or positive pressure is returned to the device and the demand valve 111 returns to its closed position. Biasing element 115 both closes demand valve 111 and returns actuator 118 to an upright or upward position. In at least one embodiment, biasing member 115 may also assist diaphragm membrane 121 to return to an upward position.

Outlet 130 is formed in back plate 103 and configured to couple to a mouthpiece through a hose or pipe (both not shown).

FIG. 3 also shows a threaded tube 131. Threaded tube 131 may screws inside first member 113 and press against the flat, outward face of stem 114 to inhibit gas from entering device 100. In at least one embodiment, threaded tube 131 may be one of the elements of device 100 that can be used to adjust the cracking pressure.

In at least one embodiment, an alternate path is provided in intake block 112 for gases to enter chamber 108. Referring to FIGS. 4A-4C, shown therein is an aperture 134 configured to receive a fastener 132. Aperture 134 is formed by drilling a hole in the intake block 112 and connecting the hole to respective holes 135, 136 for each of the demand valves 111. This is shown in FIG. 4C. Fastener 132 is used to seal the side hole 134 connecting the valve holes 135, 136 to an alternate gas path 137.

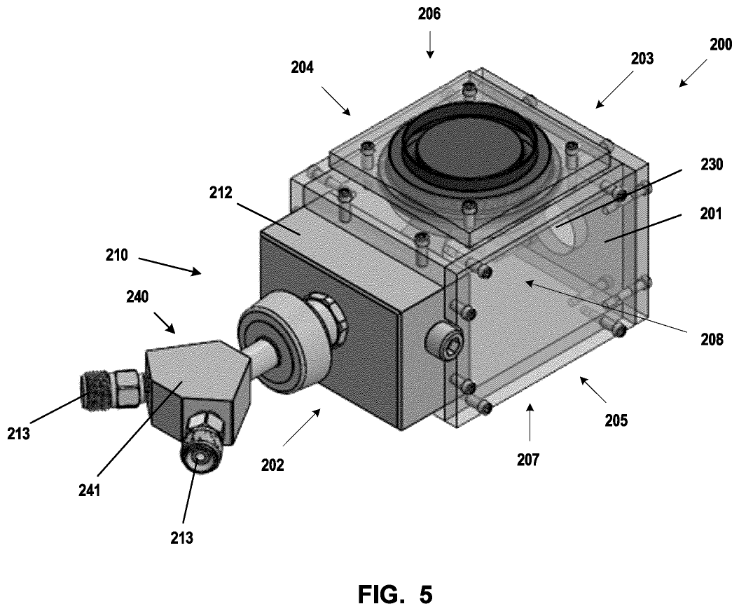

Turning to FIGS. 5-8, the embodiment shown therein is an alternative embodiment of device 200 that has the same overall function as device 100. Demand valve assembly 210 of device 200 includes a single demand valve 211 extending from the intake block 212 as well as an input assembly 240. Input assembly 240 receives two or more gases from respective sources via first members 213, combines the gases together within an input block 241 and then supplies the gas(es) from the input block 241 into the intake block 212 and chamber 208.

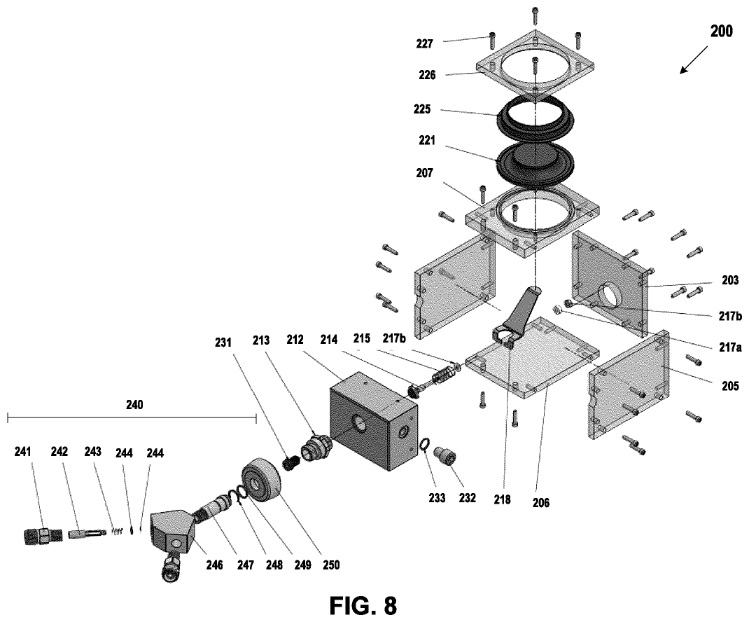

In FIG. 8, input assembly 240 includes two or more one-way valves 241 that provide for gas to only flow inwardly towards the device 200. To provide for one-way flow, valves 241 include a stem 242, biasing member 243 and one or more sealing members 244 (e.g. o-ring, etc.). Each one-way valve 241 couples to a preliminary chamber 246 that receives gases from each one-way valve 241 and provides for the gases to mix with each other before being provided to the chamber 208 of device 200.

Pathway member 247 couples to an outlet of preliminary chamber 246 and carries the mixed gases towards the first member 213 and intake block 212. Additional component such as but not limited to sealing members 248, 249 as well as ring 250 may be provided for sealingly coupling the input assembly 240 to the first member 213.

While the applicant's teachings described herein are in conjunction with various embodiments for illustrative purposes, it is not intended that the applicant's teachings be limited to such embodiments as the embodiments described herein are intended to be examples. On the contrary, the applicant's teachings described and illustrated herein encompass various alternatives, modifications, and equivalents, without departing from the embodiments described herein, the general scope of which is defined in the appended claims.

What is claimed is:

1. A device for delivering at least one gas to a user, the device comprising:
a body defining:
a chamber to receive the gas from a source;
an inlet leading into the chamber; and
an outlet for the user to draw the gas from the chamber;
a demand valve assembly configured to supply the gas to the chamber via the inlet, the demand valve assembly comprising:
an intake block secured to the body;
at least one demand valve coupled to the intake block and configured to control the supply of gas to the chamber; and
an actuator positioned within the chamber and coupled to the demand valve, the actuator being configured to move the demand valve into an open position to supply gas to the chamber; and
a diaphragm assembly coupled to the body, the diaphragm assembly including a diaphragm membrane abutting the actuator and movable downwardly relative to the body when the user draws the gas from the chamber to move the demand valve into the open position to supply gas to the chamber.

2. The device of claim 1, wherein the demand valve assembly includes a first demand valve coupled to a first gas source to supply a first gas to the chamber and a second demand valve coupled to a second gas source to supply a second gas to the chamber, each demand valve being coupled to the actuator.

3. The device of claim 2, wherein the actuator is configured to simultaneously move each of the demand valves to their respective open positions to supply each of the gases to the chamber.

4. The device of claim 3, wherein the actuator is coupled to a stem of each of the demand valves.

5. The device of claim 4, wherein each of the demand valves includes a biasing member that biases the demand valves to a closed position.

6. The device of any one of claims 1-5, wherein the diaphragm membrane includes a rigid portion configured to abut the actuator.

7. The device of claim 6, wherein the rigid portion is positioned in a center of the diaphragm membrane.

8. The device of claim 7, wherein the actuator abuts the rigid portion of the diaphragm membrane.

9. The device of any one of claims 1-8, wherein the diaphragm membrane is made of a polymeric material configured to provide the demand valve with a cracking pressure suitable to inhalation capabilities of a user with respiratory distress.

10. The device of any one of claims 9, wherein the diaphragm membrane is made of a polymeric material configured to provide for the demand valve to have a cracking pressure in a range of about −1 cm H₂O to about −3 cm H₂O.

11. The device of claim 1 further comprising an input assembly configured to receive two or more gases from respective sources, the input assembly being coupled to the demand valve assembly for providing the gases to the chamber.

12. The device of claim 11, wherein the input assembly comprises a preliminary chamber configured to receive two or more gases from respective sources and provide for the two or more gases to mix with each other prior to being provided to the chamber.

13. The device of claim 12, wherein the input assembly comprises two one-way valves, each one-way valve for receiving one of the two or more gases and inhibiting the two or more gases from exiting the preliminary chamber.
